(19) United States
(12) Patent Application Publication
(10) Pub. No.: US 2016/0304828 A1
(43) Pub. Date: Oct. 20, 2016

(54) Automated Protein Production And Cell Lysing

(71) Applicant: Terumo BCT, Inc., Lakewood, CO (US)

(72) Inventors: Briden Ray STANTON, Highlands Ranch, CO (US); Ashley L. BECKWITH, Monument, CO (US); Hilary R. HAWS, Golden, CO (US); Dalton A. NOREN, Ft. Collins, CO (US); Joshua PICKRELL, Severance, CO (US)

(21) Appl. No.: 15/130,816

(22) Filed: Apr. 15, 2016

Related U.S. Application Data
(60) Provisional application No. 62/148,627, filed on Apr. 16, 2015.

(51) Int. Cl. C12M 1/36; C12M 1/34; C12N 1/06; C07K 1/14; C12M 1/00

(52) U.S. Cl. CPC C12M 41/48; C07K 1/145; C12M 47/06; C12N 1/066; C12M 41/12; C12M 47/10

(57) ABSTRACT

Embodiments of methods and systems are described for automating protein production from genetically modified microorganisms. Embodiments provide for growing modified microorganisms to generate protein, releasing the protein and recovering the protein automatically. Also described are embodiments for lysing cells that may be used to as part of an automated system to recover the protein after generation by the microorganisms.

### CROSS-REFERENCE TO RELATED PATENT APPLICATION(S)

This patent application claims priority to U.S. Provisional Patent Application No. 62/148,627 filed Apr. 16, 2015 entitled “AUTOMATED CELL LYSING,” which is hereby incorporated by reference in its entirety as if set forth herein in full.

### BACKGROUND

Within the biological sciences there exist protocols involving the use of E. coli (or other host organisms) to produce proteins by expression of a specific gene. Scientists are able to insert the gene of interest into a microorganism, for example a bacteria cell (e.g., Escherichia coli) and use the exponential growth properties of the bacteria to greatly increase the amount of recombinant protein that is produced. The protein may be produced using batch or continuous feed processes.

In order to recover the protein or other material for which the gene of interest codes, the cells may be lysed to release the protein or material. The processes for lysing the cells and recovering the protein, or other material, from the lysed cells conventionally involve steps that make the process inefficient.

Embodiments of the present invention have been made in light of these and other considerations. However, the relatively specific problems discussed above do not limit the applicability of the embodiments of the present invention.

### SUMMARY

The summary is provided to introduce aspects of some embodiments of the present invention in a simplified form, and is not intended to identify key or essential elements of the claimed invention, nor is it intended to limit the scope of the claims.

Embodiments may relate to methods and systems for automating protein production from genetically modified microorganisms. In embodiments, a system is provided that includes at least a cell growth system for growing genetically modified microorganisms in a culture medium; an optical system for determining a concentration of microorganisms in the culture medium; a separation system for separating at least a portion of the microorganisms from other liquid components; a fluid circulation system that provides fluid communication among the various systems; and at least one processor configured to perform one or more steps. In some embodiments, the system may further include a lysing system for lysing the microorganisms to release protein from the microorganisms and a protein recovery system for recovering the released protein.

The steps may include controlling the growth conditions under which the microorganisms in the growth system are grown. After microorganisms have grown, controlling a first flow of fluid (including microorganisms) from the growth system to the optical system to determine an extent of microorganism growth. The method may further involve controlling a second flow of fluid to a separation system to separate microorganisms from other components. A third flow of fluid is controlled to move fluid (including liquid) from the separation system to a first container. A fourth flow is controlled to move fluid including concentrated microorganisms from the separation system. In some embodiments, the system may further provide for lysing the concentrated microorganisms. In yet other embodiments, the system may further provide for a protein recovery system. The protein recovery system may include a column for binding the protein (released by the lysing) to a column material. After binding of the protein, the protein may be extracted from the column material and recovered.

Other embodiments may relate to methods and systems for automatically lysing cells (e.g., cells of microorganisms) to collect proteins of the lysed cells. Embodiments may provide for a system that may include a manipulating device and a lysing chamber. In embodiments, the lysing chamber may include beads. The manipulating device may be connected to the lysing chamber and may manipulate the chamber, such as by rocking, stirring, vibrating, turning, agitating, or otherwise moving the lysing chamber. The movement of the chamber creates shear forces, and collisions between beads and cells that result in lysing of the cells. The lysing may be performed automatically without the need for manual input by an operator. In embodiments, the system may be closed and fluid may flow through portions of the lysing system without exposure to the atmosphere.

### BRIEF DESCRIPTION OF THE DRAWINGS

Non-limiting and non-exhaustive embodiments are described with reference to the following figures.

FIG. 1 illustrates an embodiment of a protein production system in accordance with embodiments.

FIG. 2 illustrates a flow chart of a method of producing protein in accordance with one embodiment.

FIG. 3 illustrates a chart of microorganism growth over time.

FIG. 4 illustrates a lysing system in accordance with embodiments.

FIG. 5 illustrates a schematic of a second lysing system in accordance with embodiments.

FIG. 6 illustrates a third lysing system in accordance with embodiments.

FIG. 7 illustrates a schematic of the lysing chamber shown in FIG. 6.

FIG. 8 illustrates a flow chart of a method of lysing cells according to an embodiment.

FIG. 9 illustrates example components of a basic computer system upon which embodiments may be implemented.

### DETAILED DESCRIPTION

The principles of the present invention may be further understood by reference to the following detailed description and the embodiments depicted in the accompanying drawings. It should be understood that although specific features are shown and described below with respect to detailed embodiments, the present invention is not limited to the embodiments described below.

Reference will now be made in detail to the embodiments illustrated in the accompanying drawings and described below. Wherever possible, the same reference numbers are used in the drawings and the description to refer to the same or like parts.

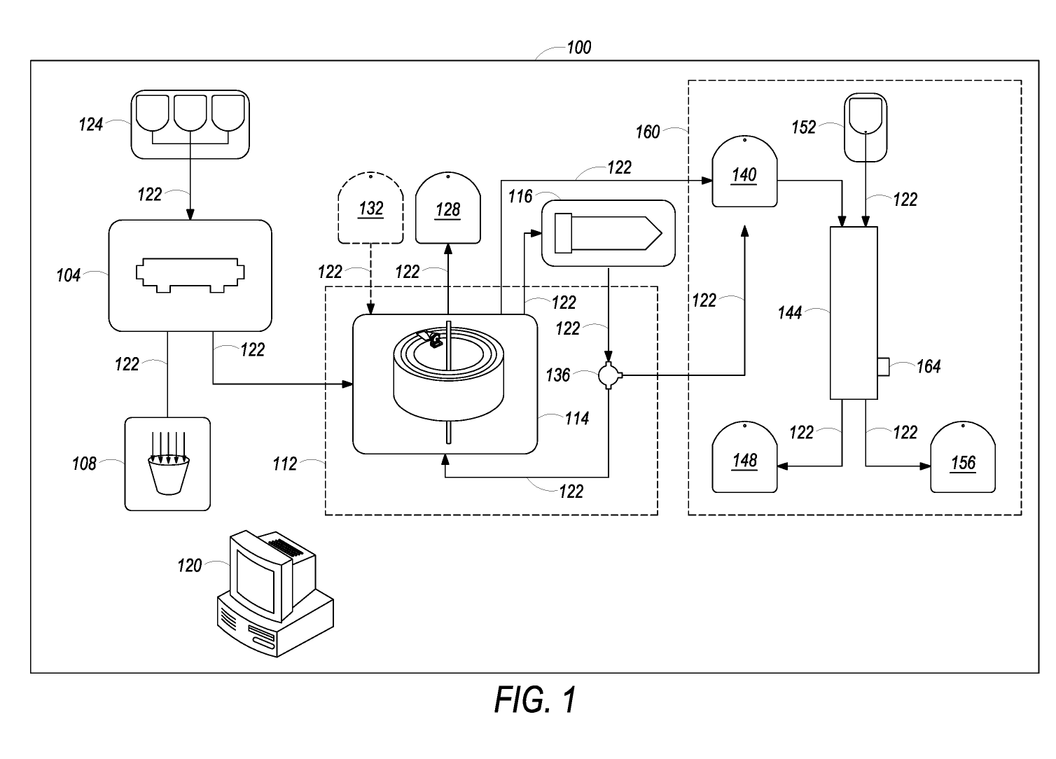

FIG. 1 illustrates an automated protein production system 100 that may implement embodiments of the present invention. System 100 may provide a single system that is capable of automatically generating and recovering protein expressed by microorganisms. In some embodiments, the microorganisms are genetically modified with a DNA sequence that codes for a specific protein of interest. As described below system 100 may be used to grow the modified microorganisms and collect the protein of interest, which may be used for research or other purposes, e.g., therapeutic purposes.

System 100 includes, among other features, a cell growth system 104, an optical system 108, a separation system 112, a lysing system 116, and a computing system 120. Also shown is a fluid conduit system 122 that fluidly connects various portions of system 100. Although not shown in detail, fluid conduit system 122 may include a number of features non-limiting examples including pumps, valves, conduits (e.g., tubing, piping, couplings, etc.), flow monitors/sensor, and/or pressure regulators. It is noted that although particular components are discussed with respect to system 100, other embodiments may not include all of the components shown in FIG. 1 or described below.

In operation, microorganisms (which have been modified to express a particular desired protein) are placed in cell growth system 104 in order to grow the microorganisms. In addition, culture medium that includes the necessary nutrients is also added to cell growth system 104.

The microorganisms grown in cell growth system 104 may be any appropriate microorganisms for expressing a desired protein. Some non-limiting examples of microorganisms that may be grown in cell growth system 104 include, Escherichia coli, Corynebacterium, Pseudomonas fluorescens, Saccharomyces cerevisiae, Pichia Pastoris, Filamentous fungi, and Baculovirus-infected cells.

In some embodiments, microorganisms, culture media, reagents, nutrients, etc. may be added manually, such as by an operator to cell growth system 104. In other embodiments, a delivery system 124 may be configured to automatically add these to cell growth system 104. Although not shown, the delivery system may include a number of different features such as reservoirs (for storing various reagents), pumps, valves, fluid conduits (tubing), measuring devices, and/or sensors.

Computer system 120 may be connected to one or more components/subsystems of system 100. Although system 120 is shown in FIG. 1 as generally connected to system 100, it may be wired or wirelessly connected to one or more components/subsystems of system 100 individually. Alternatively, it may be wired or wirelessly connected to a central connection that is also connected to one or more components/subsystems of system 100.

As described in greater detail below, computer system 120 may include logic that performs various steps including: receiving data/signals from one or more components/subsystems of system 100; sending data/signals to on one or more components/subsystems of system 100; and/or displaying data/signals to an operator. Further, although computer system 120 is illustrated as a single system, it may in embodiments be more than one system. As one example, the system may be a distributed system, with more than one central processing unit, each central processing unit controlling different aspects of system 100.

In embodiments, computer system 120 may be configured to monitor growth conditions in cell growth system 104. In these embodiments, computer system 120 may communicate with various sensors in growth system 104 that provide data regarding conditions non-limiting examples including temperature, pH, nutrient levels, oxygen levels, and carbon dioxide levels. In response to detecting the conditions, computer system 120 may prompt an operator to add material to cell growth system 104, or control delivery system 124 to automatically deliver additional material to cell growth system 104 to maintain optimal growth conditions for the microorganisms. As another example, cell growth system 104 may in embodiments include a heating source (e.g., resistive coils) that may be controlled by computer system 120 to maintain the temperature of the system at an optimal temperature for growing the microorganisms. In one embodiment, the growth conditions are optimized for growing Escherichia coli and/or genetically modified Escherichia coli.

At predetermined periods of time, or alternatively in response to certain conditions in cell growth system 104 (e.g., temperature, pH, nutrient levels, oxygen levels, and carbon dioxide levels), computer system 120 may provide for a portion of fluid conduit system 122 to deliver a volume of fluid to the optical system 108 from the cell growth system 104. The optical system 108 may be used to determine a concentration of microorganisms as well as other data, such as solution turbidity. It may be useful in embodiments to obtain information regarding the concentration of microorganisms to determine the optimal time to initiate protein expression. In embodiments, optical system 108 may include one or more cameras, photodetectors, LED's, reflectors, logic, wavelength photodiodes, or cuvettes.

After predetermined conditions are met, e.g., concentration of microorganisms in cell growth system 104 or a predetermined period of time, computer system 120 may initiate the expression of a protein by the microorganisms growing in cell growth system 104. In embodiments, this may involve prompting an operator to add a reagent, such as Isopropyl β-D-1-thiogalactopyranoside (IPTG) to cell growth system 104 to initiate protein expression. In some embodiments, the reagent may be added to cell growth system 104 automatically without operator intervention using delivery system 124.

After the microorganisms in cell growth system 104 have expressed the protein, e.g., after a predetermined period of time, computer system 120 may initiate flow of a cell suspension fluid (with microorganism, culture media, nutrients, waste products, etc.) from cell growth system 104 to separation system 112 using part of conduit system 122. The cell suspension may be pumped from cell growth system 104 to separation system 112, which as described below may include a number of features for separating components of the cell suspension.

In the embodiment shown in FIG. 1, the separation system 112 includes centrifuge 114. Centrifuge 114 may be used to separate the microorganisms that have expressed the protein from other components in the cell suspension. As a result, fluid from cell growth system 104 may be separated into a liquid component, e.g., supernatant, and a concentrated microorganism component. The liquid may be directed to a container 128 for storage, disposal, or recycling, in a different process.

The concentrated microorganisms may in some embodiments be directed from centrifuge 114 to lysing system 116. The step of directing the separated microorganisms to lysing system 116 may be preceded by adding some additional liquid to the separated microorganisms to allow the separated microorganisms to more easily flow from the centrifuge 114 to lysing system 116. The additional liquid may be added, in some embodiments, automatically, using delivery system 132.

Lysing system 116 may provide features that lyse the cell membranes of the microorganisms. In embodiments, lysing system 116 may utilize ultrasonic energy, temperature, chemicals, shear forces, osmotic forces and/or other lysing techniques to lyse the microorganisms. As one non-limiting example, the lysing system may utilize ultrasonic transducers to create a standing wave that shears the microorganisms. In another example, lysing system 116 may deliver a chemical that disrupts the cell membranes of the microorganisms.

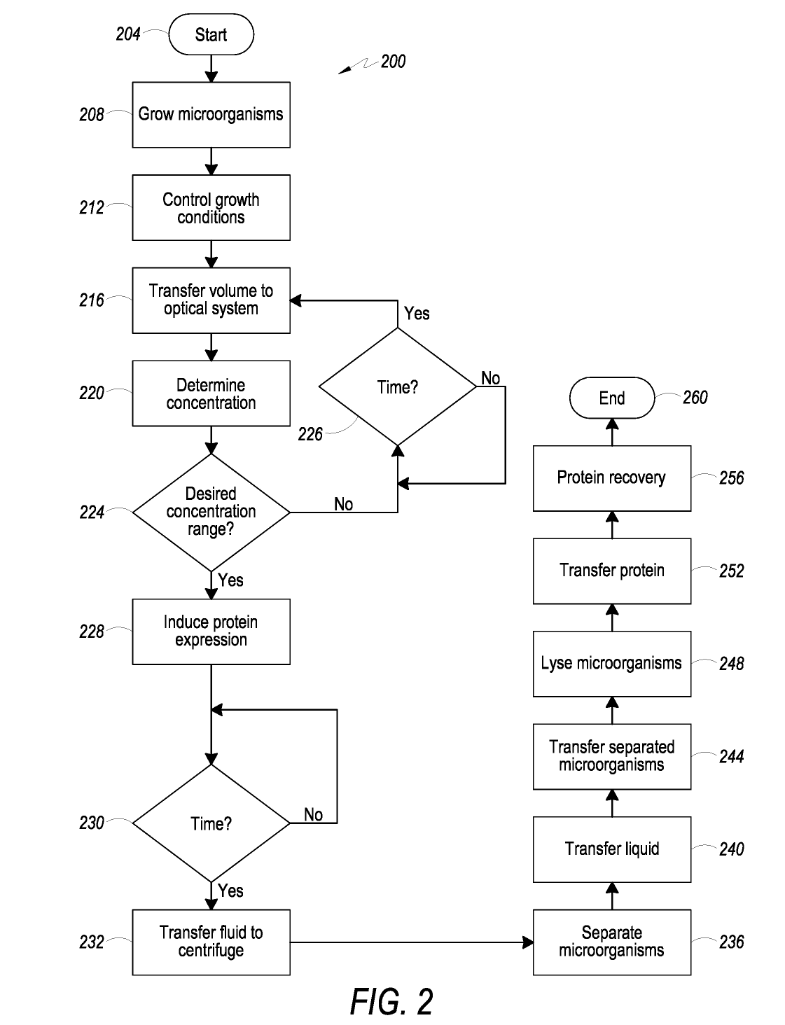

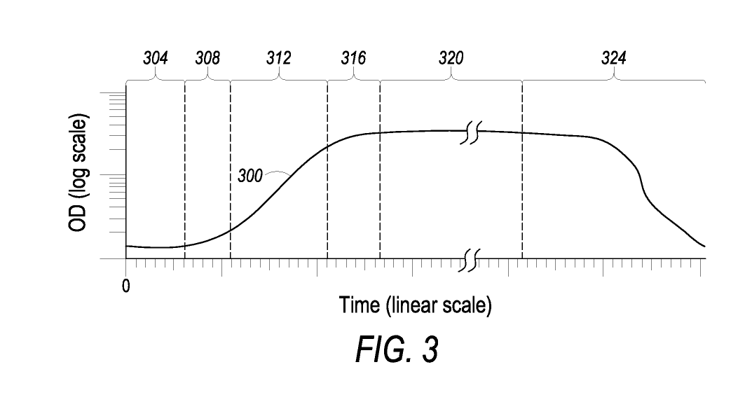

Embodiments of some lysing systems, and/or their component parts, are described below with reference to FIGS. 2 and 3. As described in greater detail below, some embodiments of lysing systems may utilize shear forces and mechanical disruption of cell membranes to lyse the microorganisms.

After lysing, the cellular protein is in a fluid in the lysing system 116. The fluid with the protein released from the microorganisms may be optionally directed from lysing system 116 to separation system 112 for separating the protein from other material, e.g., cellular components such as membranes. In embodiments, a fluid stream may be directed from lysing system 116 to filter 136 in separation system 112. In some embodiments, beads may be used during the lysing process (described in greater detail below), which may be removed in filter 136 before further processing of the protein. In other embodiments, filter 136 may, in addition to beads, remove other material such as cell membranes or other material.

In some embodiments, filtered cellular protein may then be directed to a protein recovery system 160 for recovering the desired cellular protein. However, alternatively, the filtered protein may be directed from filter 136 to centrifuge 114. In centrifuge 114, additional material may be separated from the desired protein by centrifugation. For example, if the lysing system uses beads, any remaining beads may be separated. The further purified protein may then be directed to protein recovery system 160.

In some embodiments, protein recovery system 160 may include container 140 for storing fluid from filter 136 and/or centrifuge 114. Other embodiments may not include container 140 and may direct fluid from filter 136 and/or centrifuge 114 to column 144. Column 144 may be used to bind the protein and recover the protein, such as through chromatography. Protein may flow from container 140 (or directly from filter 136 or centrifuge 114) through a portion of fluid conduit system 122 to column 144. Alternatively, the protein may flow directly from filter 136 or centrifuge 114 to column 144.

At least a portion of the protein may bind to material in column 144 as it flows through column 144. Liquid containing a depleted amount of protein may flow from column 144 to a container 148. In some embodiments, instead of flowing into container 148, the liquid may be re-circulated through column 144 so that additional protein may be bound to material in column 144 before it is directed to container 148 for disposal or reuse. In embodiments, sensor(s) 164 may generate a signal indicating that the binding of the protein to the material in the column is complete (e.g., liquid from container 140 has finished flowing through column 144). In embodiments, the sensor(s) 164 may include one or more of optical sensor, flow meters, transducers, temperature sensors, etc.

Finally, after the protein is bound to material in column 144, an extracting liquid may be flowed into column 144 from delivery system 152 to extract the protein bound to the material. In embodiments, computer system 120 may automatically control delivery system 152 and control the flow of extraction fluid flowed through column 144. Sensor(s) 164 may for example generate a signal indicating that a particular volume of extraction liquid has flowed through column 144 providing a level of confidence that the extraction of the protein may be relatively complete (e.g., as much protein as practically possible has been extracted from the column material). In response to a signal from sensor 164, computer system 120 may signal the delivery system 152 to stop flow of extracting liquid through column 144. The extracted protein and the extracting liquid may be collected and stored in container 156.

It is noted that the present invention is not limited to being implemented in the system 100 shown in FIG. 1 and described above. In other embodiments, the system may include fewer than the features shown in FIG. 1, while in yet other embodiments, system 100 may include more than the features shown in FIG. 1. As one example, the fluid conduit system 122 may include valves, pumps, or other flow controlling devices that may be controlled by computer system 120. As another example, although column 144 is shown as a single column, in other embodiments, it may be a series of columns, such as a series of affinity chromatography columns.

In embodiments, system 100, and/or portions of system 100, may be implemented in a disposable or reposable component(s). For example, in embodiments, growth system 104 may include an incubator that is configured to fit a disposable growth chamber. The growth chamber may be connected to a disposable vessel through tubing. The disposable vessel may be configured to fit in centrifuge 114. Further, the disposable vessel may by connected to tubing (serving as conduit system 122) that connects to a disposable lysing chamber in lysing system 116. The disposable lysing chamber may be configured to connect to a manipulation device (e.g., vortexer 158) that aids in lying material within the disposable lysing chamber. The disposable lysing chamber may be connected to a disposable filter 136. Additional tubing may connect filter 136 to portions of protein recovery system 160, such as for example, column 144, which may be disposable. There may be additional tubing, connections, containers, or other features that are part of recovery system 160.

System 100 in embodiments provides a “closed system” that allows cells (e.g., genetically modified cells) to be grown, activated to generate a protein, separated from other material, lysed to release protein, and the protein recovered without exposure to the atmosphere. Moreover, computer 120 may be configured to send signals to various components of system 100 to automatically perform any necessary steps. That is, in embodiments, system 100 may provide a fully automated, closed system that allows an operator to load a volume of cells into the cell growth system 104 and after a period of time, obtain a volume of desired protein in container 156.

The terms “automated”; “automatically;” and similar terms are intended to mean, in this patent application, without the need for an operator's manual input. That is, a particular step or series of steps may be performed without the need for a person to perform any action with the exception, in some embodiments, of initiating the start of a step or series of steps.

FIG. 2 illustrates a flow 200 of a method of producing protein in accordance with one embodiment. In some embodiments, the steps of flow 200 may be performed by one or more features of system 100 (FIG. 1), e.g., computer system 120. The description of flow 200 below may be described as being performed by one or more features of system 100. This is done merely for illustrative purposes, and flow chart 200 is not limited to being performed by any specific device or component(s). The steps may be performed by other features not shown in the figures or described herein but still be within the scope of the present disclosure.

Flow 200 begins at step 204 and passes to step 208 where microorganisms are grown. Step 208 may involve various sub-steps such as adding microorganisms, e.g., genetically modified microorganisms, to a cell growth system such as system 104. Other sub-steps may involve adding reagents, nutrients, or other additions to the growth system with the microorganisms.

Flow 200 passes from step 208 to step 212, where growth conditions of the microorganisms are controlled. For example, the growth conditions may include one or more of temperature, reagent concentrations, pH, nutrient concentrations, and/or gas concentrations. Step 208 may be performed by for example a computer system such as system 120, which may for example control features such as heaters, aerators/gas transfer devices, nutrient delivery systems, and or reagent delivery systems. The computer system may receive information or data from various sensors of the cell growth system 104 and in response to the data, perform actions (e.g., actuate valves, turn on/off heating elements, agitate fluid, etc.) to maintain the growth conditions of the microorganisms within a predetermined range for each parameter that optimizes the growth of the microorganisms. Specific embodiments provide for the growth conditions to be optimized for growing Escherichia coli and/or genetically modified Escherichia coli.

Flow 200 then passes to step 216 where a sample of fluid is transferred to an optical system. In embodiments, the sample of fluid, with the culture medium and the microorganisms, may be periodically delivered to an optical system (e.g., system 108) until a particular set of conditions is met.

After step 216, at step 220, a determination is made as to the concentration of microorganisms in the sample. At decision 224 a determination may be made as to whether a concentration of microorganisms is within a predetermined range. Without being bound by theory, it is believed that at particular concentrations along a growth curve, initiating protein expression will maximize the amount of protein that will be expressed and ultimately collected.

Referring to FIG. 3, an example of a microorganism, e.g., bacteria, growth curve 300 is illustrated as optical density (OD) over time. As curve 300 illustrates, initially there is a lag phase 304 where the microorganisms do not grow significantly. Phase 304 is followed by a starting phase 308 where microorganism growth begins to accelerate. At phase 312, microorganism growth is exponential, which is followed by a slow-down growth phase 316. Growth levels out during a stationary phase 320, which is followed by a die-off phase 324. As may be appreciated by those of skill in the art, optimal protein production may depend, at least in part, upon the timing of protein expression during the growth of the microorganisms. The predetermined concentration range used at decision 224 may depend upon the protein being produced.

If at decision 224 it is determined that the concentration determined at step 220 is not at a desired concentration, e.g., the microorganism growth is in the lag phase and not the exponential growth phase, flow passes to decision 226 where a determination is made whether a predetermined period of time has passed since step 216 was performed. If the predetermined period of time has not passed, determination 226 is repeated. If the predetermined period of time has passed, flow passes back to step 216 where another volume of fluid is transferred and steps 220, decisions 224 and 226 are repeated.

If at decision 224 it is determined that the concentration determined at step 220 is at a desired concentration, e.g., the microorganism growth is in the exponential growth phase, flow 200 passes to step 228 where protein expression is induced. In embodiments, step 228 may involve automatically adding a reagent, such as IPTG to the growth system, or making a change to the cell growth system, culture medium, and/or microorganisms. In other embodiments, step 228 may provide some prompt to an operator that may then add the reagent or provides some other change that initiates the expression of protein.

Flow 200 then passes from step 228 to decision 230 where a determination is made whether a predetermined period of time has passed. As may be appreciated, the predetermined period of time may be selected for optimal microorganism growth and, since protein expression was initiated at step 228, also optimal protein production. If the predetermined period of time has not elapsed flow 200 pauses.

After the predetermined period of time has elapsed, flow passes from decision 230 to step 232 where fluid is transferred to a centrifuge (e.g., centrifuge 114) of a separation system 112. Step 232 may be performed by a computer system such as system 120 that controls a circulation system to move fluid with the microorganisms that have expressed the protein and the culture medium, from the cell growth system to a separation system (e.g., separation system 112).

At step 236 the microorganisms that have expressed the protein are then separated from liquid, e.g., culture medium. At step 240 the separated liquid is transferred at step 236, such as by flowing the liquid into a container for storage and later disposal, reuse, or repurpose.

At step 244 the microorganisms separated at step 236 are transferred, such as by flowing the separated microorganisms into one or more of a container, filter, or lysing system. In the embodiment shown in FIG. 2, the separated microorganisms may be transferred to a lysing system.

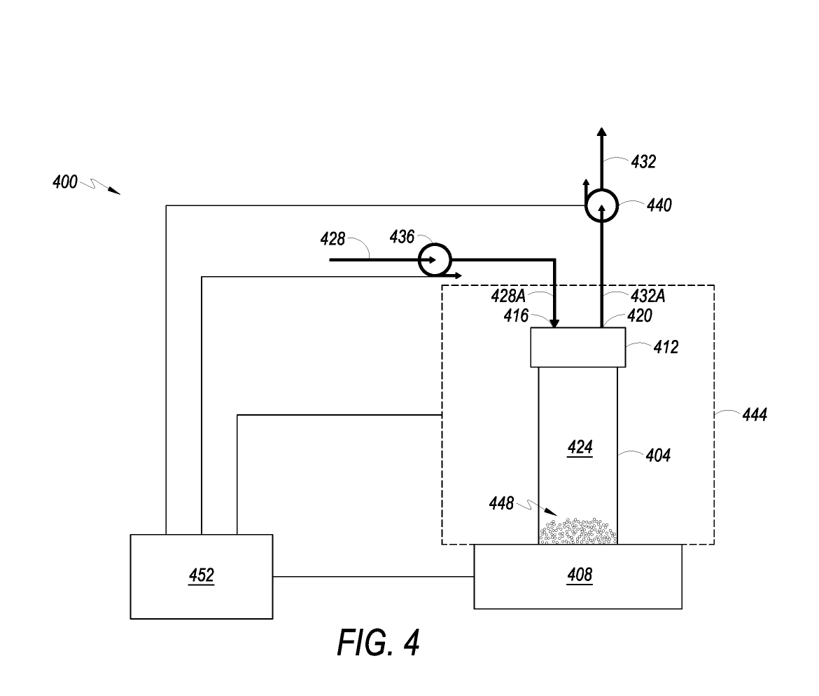

After step 244, at step 248, the microorganisms separated at step 236 are lysed in a lysing system. Step 248 may be performed using any appropriate feature. In some embodiments, a lysing system such as lysing system 160 may be used. When the microorganisms are lysed at step 248, the cell membranes of the microorganisms are disrupted and the protein is released allowing it to be recovered. The lysing step 248 may utilize any appropriate lysing technique such as chemical, temperature, mechanical (e.g., shear forces), osmotic, and/or combinations thereof. Examples of some lysing systems consistent with embodiments are shown in FIGS. 4-7 and described below.

At step 252, after the lysing step 248, a fluid with the protein released during the lysing step 248 is transferred from the lysing system. Step 252 may involve transferring the fluid to a storage container or to another system for additional processing. In the embodiment shown in FIG. 2, the fluid with the protein may be transferred to a protein recovery system.

After step 252, flow passes to step 256 where the protein is recovered. Step 256 may involve a number of sub-steps. In embodiments, the flow of protein may be directed to a filter to remove cell membranes and other material before the protein flow is directed to a container for storage. In other embodiments, step 252 may involve use of a centrifuge (e.g., centrifuge 114 of separation system 112) to separate cell membranes or other material from the protein, which may then be directed to a container for storage. In yet other embodiments, step 252 may involve directing the flow of protein, after a separation process to remove cell membranes and/or other material from the protein, to a column to further separate the protein. In these embodiments, a column (e.g. column 164) may be used to bind the protein to column material. In later steps, the bound protein may then be extracted using an extracting liquid. Flow 200 then ends at 260.

It is noted that although flow 200 has been described above with various steps in particular order, the present invention is not limited thereto. In other embodiments, the various steps and sub-steps may be performed in a different order, in parallel, partially in the order shown in FIG. 2, and/or in sequence as shown in FIG. 2. Also, the description above indicating that the step or sub-steps are performed by particular features or structures is not intended to limit the present invention. Rather, the description is provided merely for illustrative purposes. Other structures or features not described above may be used in other embodiments to perform one or more of the steps of flow 200. Furthermore, flow 200 may include some optional steps. However, those steps above that are not indicated as optional should not be considered as essential to the invention, but may be performed in some embodiments of the present invention and not in others.

FIG. 4 illustrates one embodiment of a lysing system 400 that may be used in embodiments as lysing system 116 (FIG. 1). Lysing system 400 includes a lysing chamber 404 and a manipulation device 408 connected to the lysing chamber 404 for manipulating the chamber 404. The lysing chamber 404 may include a top 412, which includes a first port 416 and a second port 420, for accessing a volume 424 of chamber 404 to transfer material into and out of volume 424. In embodiments, top 412 may be removed to access volume 424.

As shown in FIG. 4, fluid conduits 428 and 432, e.g., tubing, may be attached to ports 416 and 420 respectively. Pump 436 may be connected to fluid conduit 428 and be used to pump one or more materials, e.g., cell suspension, buffers, and/or other reagents, into volume 424 of lysing chamber 404. Pump 440 may be connected to fluid conduit 432 and be used to pump one or more materials, e.g., a fluid with cell protein and lysed cell membranes, out of volume 424 of lysing chamber 404.

A cooling system 444 may be used to cool a fluid, e.g., cell suspension, microorganisms, culture media, and/or cell protein released when cells are lysed. The cooling system 444 may in embodiments maintain the fluid at a predetermined temperature, e.g., below about 35 degrees Celsius, below about 25 degrees Celsius, below about 20 degree Celsius, below about 15 degree Celsius, or even below about 10 degrees Celsius. In other embodiments, the predetermined temperature may be above about 0 degrees Celsius, above about 2 degrees Celsius, above about 3 degrees Celsius, above about 4 degrees Celsius, or even above about 5 degrees Celsius.

Cooling system 444 is illustrated around chamber 404 as well as portions 428A and 432A of conduits 428 and 432 respectively. In some embodiments, cooling system 444 may be an insulated compartment with cold air circulating within the compartment generated by blowing air past a circulated refrigerant. Chamber 404 and portions 428A and 432A may be positioned within the compartment. In this embodiment, the cooling system 444 may be implemented as a refrigerator.

In other embodiments, the cooling system 444 may be located around only one portion of the lysing system 400. For example, in some embodiments, one or more of portions 428A and 432A may be within the cooling system 444. In some embodiments, portions 428A and 432A may be of a length that allows them to be coiled to allow the cell suspension/cell protein to be cooled for a threshold amount of time. In other embodiments, chamber 404 and manipulation device 408 may be cooled by cooling system 444.

In addition, system 400 includes a plurality of beads 448 within volume 424. The beads 448 may be used to mechanically disrupt (through collisions and/or shear forces) cell membranes, lysing the cells and releasing cell proteins one of which may be the desired protein.

Beads 448 may be of any suitable material and size. Some non-limiting examples of materials for making beads 448 include: glass, zirconia, alumina, titania, silicon carbide, and combinations thereof. The beads may in embodiments have a diameter of from about 0.1 mm to about 5.0 mm. In some embodiments, the beads 448 may have a diameter less than about 1 mm such as less than or equal to about 0.8 mm; less than or equal to about 0.7 mm; less than or equal to about 0.6 mm; or even less than or equal to about 0.5 mm. In some embodiments, the beads 448 may have a diameter greater than or equal to about 0.05 mm; such as greater than or equal to about 0.1 mm; greater than or equal to about 0.15 mm; greater than or equal to about 0.2 mm; greater than or equal to about 0.25 mm; or even greater than or equal to about 0.3 mm.

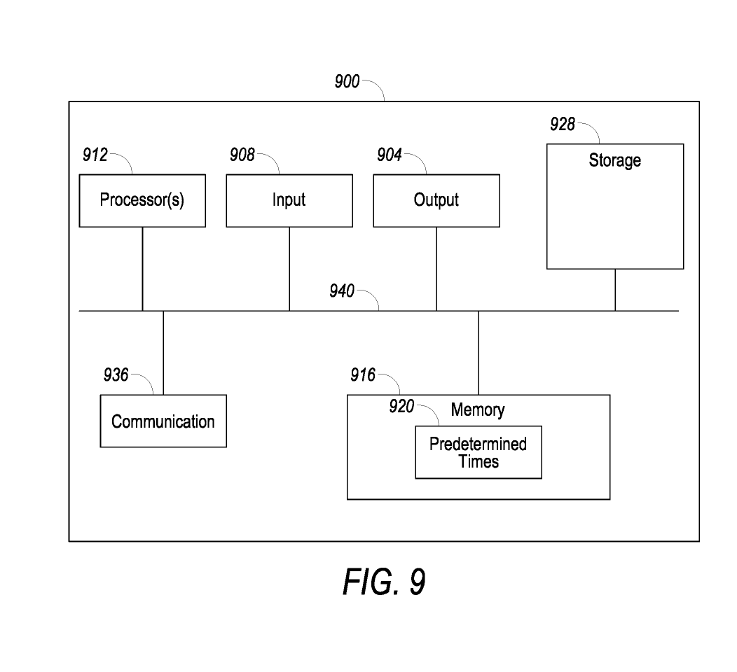

System 400 also includes a computer system 452 which includes a processor that may be configured to send signals to various parts of lysing system 400 to initiate various steps of a lysing process that may be performed automatically. As shown in FIG. 2, computer system 452 is connected to cooling system 444, pump 440, pump 436, and manipulation device 408. Computer system 452 may in embodiments include features of computer system 900 described below with respect to FIG. 9, including one or more processors.

In operation, computer system 452 may automatically perform a process of lysing cells and sending protein released from lysed cells to a protein recovery system. In embodiments, a processor in computer system 452 may be configured to send a signal to pump 436 to automatically pump a fluid (e.g., cell suspension, microorganisms, liquid, etc.) into volume 424 of the lysing chamber 404, which contains beads 448. In embodiments, the fluid may be from a separation system such as separation system 112 and include microorganisms that have been grown with the purpose of producing a particular protein.

Computer 452 may then send a signal to the manipulation device to automatically manipulate the lysing chamber 404. In embodiments, the manipulation device 408 can be a motor that activates in response to the signal sent by computer 452. The manipulation device 408 may agitate lysing chamber 404 so that the beads may collide with the cells and rupture the cell wall, releasing cellular protein. The manipulation device 408 may in some embodiments include a motor that is configured to rock, shake, stir, agitate, vibrate, rotate, or otherwise move the lysing chamber 404. In one embodiment, manipulation device is a vortexer that manipulates lysing chamber 404 to create a vortex in volume 424 of lysing chamber 404. In some embodiments, before the lysing chamber is manipulated, a buffer solution may be added to lysing chamber 404 by sending a signal to pump 436 to pump buffer solution into lysing chamber 404.

After a predetermined period of time, which may be based on the volume of cells in lysing chamber 404, the size of beads 448, and the type of manipulation device 408, computer system 452 may then send a signal to the manipulation device 408 to automatically stop the manipulation of the lysing chamber 404. In embodiments the predetermined period of time may be less than about 1 hour, less than about 45 minutes less than about 30 minutes, less than about 15 minutes, less than about 10 minutes, or even less than about 5 minutes. In other embodiments, the predetermined period of time may be greater than about 1 minute, greater than about 5 minutes, greater than about 10 minutes or even greater than about 15 minutes.

Computer system 452 may then send a signal to pump 440 to automatically pump the cell protein released from lysed cells out of the lysing chamber 404, and to a protein recovery system.

During the various steps computer system 452 may also maintain the temperature of the cell suspension or the cell protein released from the lysed cells using the cooling system 444.

As described above, system 400 may perform a lysing process with very little operator interaction. For example, an operator may simply initiate the process such as by pressing a button on computer system 452, after which system 400 can automatically perform the lysing process and deliver cell protein to a protein recovery system. Moreover, in embodiments, system 400 may perform a lysing process on a large range of volumes of fluid. For example, in embodiments, less than about 100 L of cell suspension may be lysed, while in other embodiments, less than about 75 L, less than about 50 L, less than about 25 L, or even less than about 15 L may be processed/lysed. In other embodiments, system 200 may process/lyse greater than about 2 L of cell suspension, greater than about 5 L, greater than about 10 L or even greater than about 15 L of cell suspension.

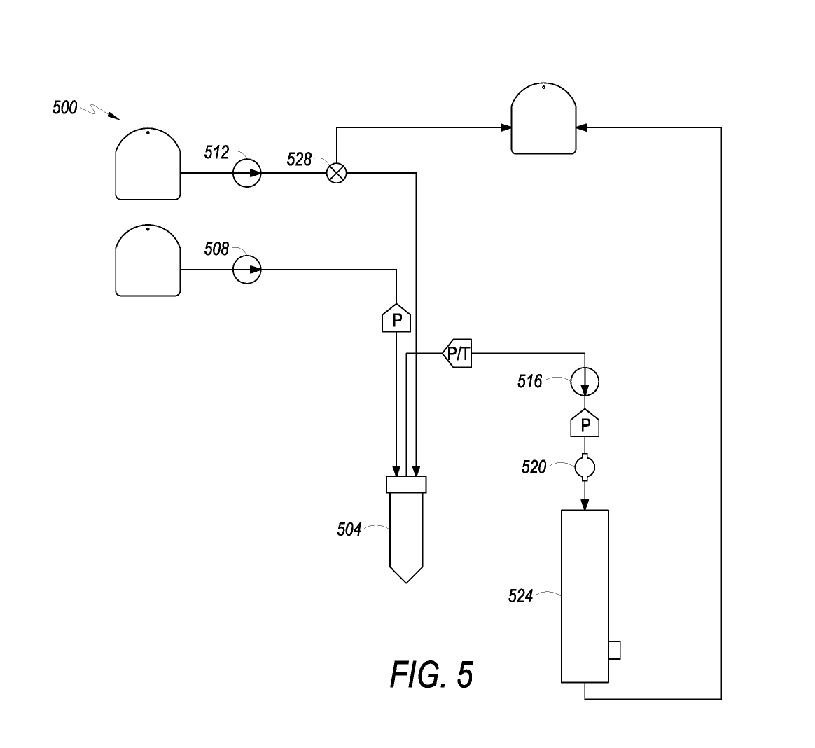

FIG. 5 illustrates a schematic of a second lysing system 500 according to embodiments. As shown in FIG. 5, system 500 includes three pumps, pump 508 for delivering a fluid, e.g., cell suspension, microorganisms, liquid, etc., into a lysing chamber 504, pump 512 for delivering a buffer into the lysing chamber 504, and pump 516 for transferring cell protein of the lysed cells/microorganisms from the lysing chamber 504 to a filter 520 and then to an affinity column 524 for protein recovery. System 500 also includes a valve 528, which may be used as a relief valve for pressure relief if the pressure is too high in the lysing chamber 504.

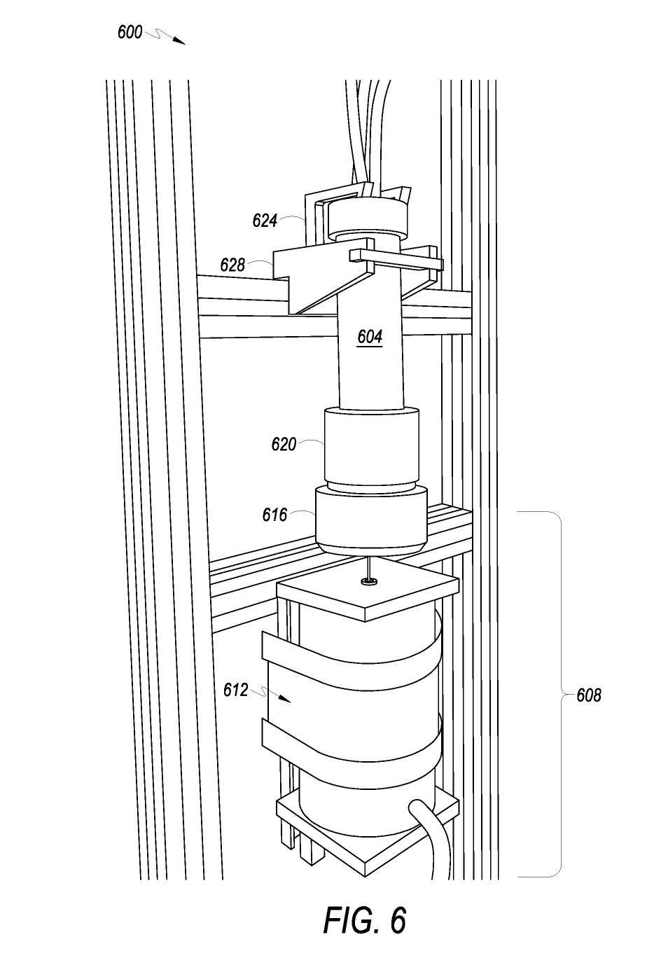

FIG. 6 is an image of a third lysing system 600 in accordance with embodiments. System 600 includes a lysing chamber 604 and a vortexer 608 (which serves as a manipulation device) connected to chamber 604 for manipulating the chamber and creating a vortex in chamber 604. Chamber 604 includes beads that collide with cells and disrupt cell membranes (through collisions and/or shear forces) when a vortex is created in chamber 604.

Vortexer 608 include a motor 612 and a motor mount 616, which in embodiments is an eccentric vortexer motor mount. A bearing 620 connects motor mount 616 with chamber 604. System 600 also includes an upper chamber support 624 and a lower chamber support 628.

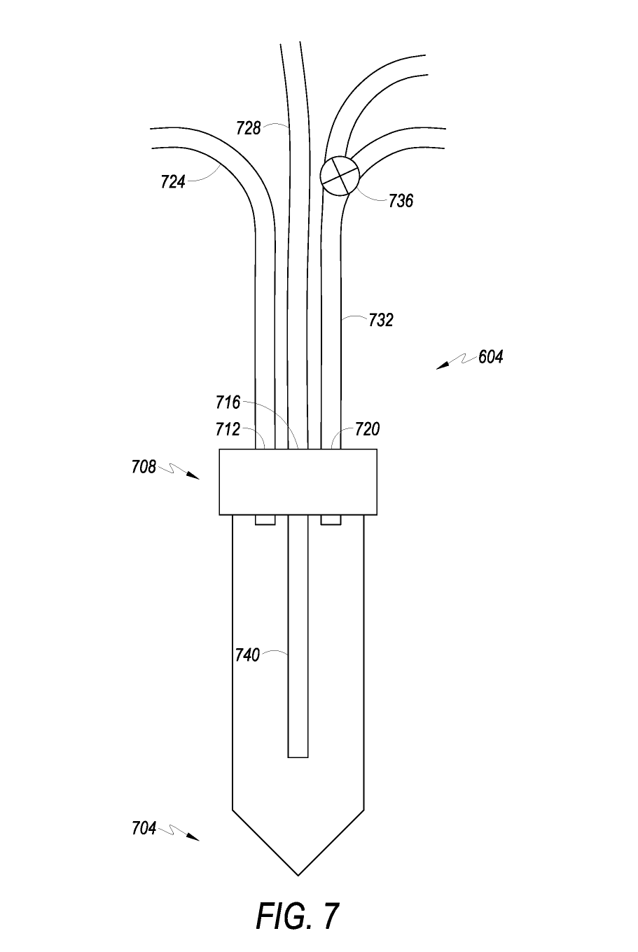

FIG. 7 illustrates a schematic of the lysing chamber 604 shown in FIG. 6. As shown in FIG. 7, chamber 604 includes a conical bottom 704, a cap 708, with three ports, 712, 716, and 720. Conduit 724 is connected to port 712, conduit 728 is connected to port 716, and conduit 732 is connected to port 720. Valve 736 is connected to conduit 732 to create two different flow paths. In a first position, valve 736 allows flow from a container with a buffer to chamber 404. In a second position, valve 736 allows flow from chamber 604 to a waste, which serves as a pressure relief path for chamber 604.

In the embodiment shown in FIG. 7, conduit 724 and port 712 serve as inlets for transferring material into chamber 604. In embodiments, a fluid, e.g., cell suspension, microorganisms, culture media, etc. may be pumped by a pump into chamber 604 through conduit 724 and port 712.

In some embodiments, a buffer solution may be transferred into chamber 604 before manipulation of chamber 604 by vortexer 608. The buffer may include a number of different materials, non-limiting examples including, additives for creating better conditions for the cell proteins that will be released after lysing, viscosity modifiers, pH modifiers, and/or other reagents. In these embodiments, valve 736 may be positioned to allow a buffer to flow from a buffer source (e.g., a container) to chamber 604 through conduit 732 and port 720.

During manipulation of chamber 604 by vortexer 608, pressure may build in chamber 604. To allow for pressure to be relieved in chamber 604, valve 736 may be positioned so that a flow path between chamber 604 and a waste container may be established. In these embodiments, any excess liquid that creates excessive pressure in chamber 604 may flow out of port 720 and conduit 736 to a waste container.

In the embodiment shown in FIG. 7, conduit 728 and port 716 serve as outlets for transferring material out of chamber 604. In embodiments, after cells have been lysed by creation of the vortex and collision of the cells with beads, the cell proteins released from the cells may be pumped by a pump out of chamber 604 through port 716 and conduit 728 to a protein recovery system. Additionally, a down tube 740 may be attached to, or be part of, conduit 728. The down tube 740, ensures that as much fluid that contains cell protein is removed from chamber 604 as possible. Accordingly, in embodiments, down tube 740 may extend further down into chamber 604 than shown in FIG. 7.

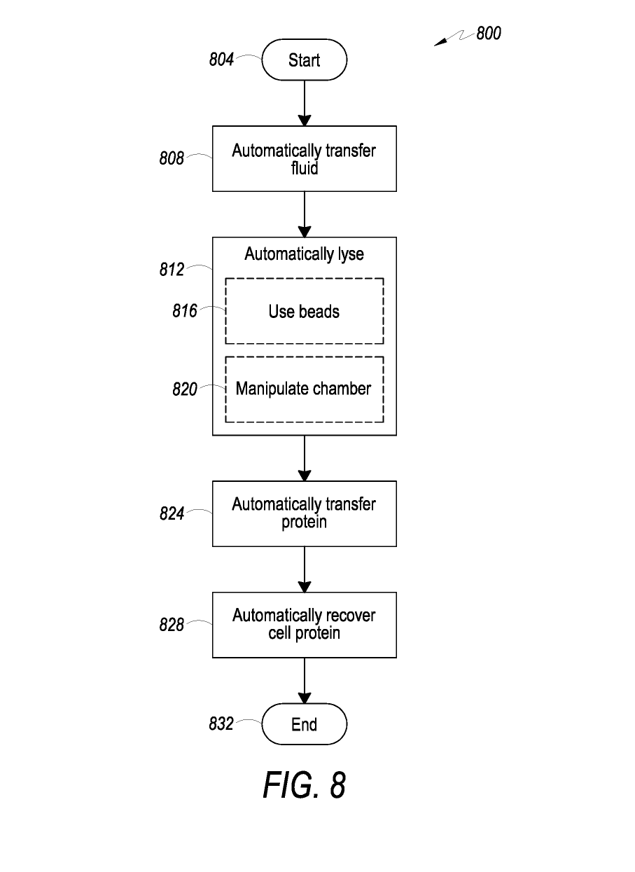

FIG. 8 illustrates a flow chart 800 of a process for lysing cells in accordance with one embodiment of the present invention. In some embodiments, the steps of flow 7800 may be performed by one or more features of systems, 100 (FIG. 1), 400 (FIG. 4), 500 (FIG. 5), 600 (FIG. 6), or 900 (FIG. 9). The description of flow 800 below may be described as being performed by one or more features of systems 100, 400, 500, 600, and/or 900). This is done merely for illustrative purposes, and flow chart 800 is not limited to being performed by any specific device or component(s). The steps may be performed by other features not shown in the figures or described above but still be within the scope of the present disclosure.

Flow 800 begins at step 804 and passes to step 808 where fluid (e.g., with cells, microorganisms, liquid, etc.) is automatically transferred into a lysing chamber. In embodiments, step 808 may be performed by a processor of a computer system, such as computer system 452 (FIG. 2) sending a signal to a pump to pump the fluid into a lysing chamber (e.g., 404, 504, or 604). Step 808 may be performed automatically. However, in embodiments, an operator may optionally initiate (e.g., press a button) to begin the step.

Flow 800 passes from step 808 to step 812 where the cells and/or microorganisms are automatically lysed. In embodiments, step 812 may be performed by a processor of a computer system sending a signal to a manipulation device.

Step 812 may in embodiments include a number of sub-steps. For example, lysing step 812 may include the use of beads 816 inside of the lysing chamber. The beads may be loaded into the lysing chamber as part of step 812 or in other embodiments; a chamber may already include beads prior to the beginning of flow 800.

Additionally, step 812 may involve sub-step 820, where the lysing chamber may be manipulated. In embodiments, sub-step 620 may involve a processor of a computer system sending a signal to a manipulation device (e.g., 408 or 608) to manipulate the lysing chamber. The manipulation device may be a motor, for example, that moves, rocks, vibrates, stirs, agitates, or otherwise manipulates the lysing chamber.

In one specific example, the manipulation device may be a vortexer (e.g., vortexer 608) and sub-step 820 may involve sending a signal to a motor of the vortexer to start. The vortexer may then manipulate the lysing chamber to generate a vortex in the lysing chamber. The vortex may create shear forces that disrupt cell membranes and lyse the cells (e.g., cells of microorganisms). In embodiments, where beads are in the lysing chamber, the vortex may also cause the beads to collide with cells, lysing the cells.

At step 824, cell protein released from the lysing step 812 are automatically transferred out of the lysing chamber. Step 824 may be performed by a processor of a computer system sending a signal for example to a pump to pump the cell protein out of the lysing chamber.

In embodiments, after, or as part of step 724, the flow of cell protein may be directed to a filter to remove cell membranes and other material before the protein flow is directed to a container for storage. In other embodiments, the flow of protein may be directed to a centrifuge (e.g., centrifuge 114) to separate cell membranes or other material from the protein, which may then be directed to a container for storage.

At step 828, the cell protein is automatically recovered. Step 828 may involve directing a flow of protein, after a separation process to remove cell membranes and/or other material from the protein, to a protein recovery system (e.g., protein recovery system 160 shown in FIG. 1). In embodiments, as part of step 828, affinity columns may be used to bind the protein. In other sub-steps of step 828, the bound protein may be extracted using an extracting liquid. Flow 800 then ends at 832.

It is noted that although flow 800 has been described above with various steps in particular order, the present invention is not limited thereto. In other embodiments, the various steps and sub-steps may be performed in a different order, in parallel, partially in the order shown in FIG. 8, and/or in sequence as shown in FIG. 8. Also, the description above indicating that the step or sub-steps are performed by particular features or structures is not intended to limit the present invention. Rather, the description is provided merely for illustrative purposes. Other structures or features not described above may be used in other embodiments to perform one or more of the steps of flow 800. Furthermore, flow 800 may include some optional steps. However, those steps above that are not indicated as optional should not be considered as essential to the invention, but may be performed in some embodiments of the present invention and not in others.

FIG. 9 illustrates example components of a basic computer system 900 upon which embodiments of the present invention may be implemented. For example, computing system 452 or 120 may incorporate features of the basic computer system 900 shown in FIG. 9. Computer system 900 includes output device(s) 904, and input device(s) 908. Output device(s) 904 include, among other things, one or more displays, including CRT, LCD, and/or plasma displays. Output device(s) 904 may also include printers, speakers etc. Input device(s) 908 may include a keyboard, touch input devices, a mouse, voice input device, scanners, etc.

Basic computer system 900 may also include a processing unit (processor) 912 and memory 916, according to embodiments of the present invention. The processing unit 912 may be a general purpose processor operable to execute processor executable instructions stored in memory 916. Processing unit 912 may include a single processor or multiple processors, according to embodiments. Further, in embodiments, each processor may be a single core or a multi-core processor, having one or more cores to read and execute separate instructions. The processors may include general purpose processors, application specific integrated circuits (ASICs), field programmable gate arrays (FPGAs), and other integrated circuits.

The memory 916 may include any tangible storage medium for short-term or long-term storage of data and/or processor executable instructions. The memory 916 may include, for example, Random Access Memory (RAM), Read-Only Memory (ROM), or Electrically Erasable Programmable Read-Only Memory (EEPROM). Other storage media may include, for example, CD-ROM, tape, digital versatile disks (DVD) or other optical storage, tape, magnetic disk storage, magnetic tape, other magnetic storage devices, etc.

Storage 928 may be any long-term data storage device or component. Storage 920 may include one or more of the devices described above with respect to memory 916. Storage 928 may be permanent or removable.

Computer system 900 also includes communication devices 936. Devices 936 allow system 900 to communicate over networks, e.g., wide area networks, local area networks, storage area networks, etc., and may include a number of devices such as modems, hubs, network interface cards, wireless network interface cards, routers, switches, bridges, gateways, wireless access points, etc.

The components of computer system 900 are shown in FIG. 9 as connected by system bus 940. It is noted, however, that in other embodiments, the components of system 900 may be connected using more than a single bus.

In embodiments, computing systems 120 (FIG. 1) and 452 (FIG. 4) may include aspects of system 900. In these embodiments, memory 916 may store predetermined times 920, which may be used to determine how much time to lyse cells, allow microorganisms to grow, allow proteins to be generated after protein generation is induced, etc.

It will be apparent to those skilled in the art that various modifications and variations can be made to the methods and structure of the present invention without departing from its scope. Thus it should be understood that the invention is not be limited to the specific embodiments or examples given. Rather, the invention is intended to cover modifications and variations.

While example embodiments and applications of the present invention have been illustrated and described, it is to be understood that the invention is not limited to the precise configuration and resources described above. Various modifications, changes, and variations apparent to those skilled in the art may be made in the arrangement, operation, and details of the methods and systems of the present invention disclosed herein without departing from the scope of the invention.

What is claimed is:

1. A system for lysing cells, the system comprising:
a lysing chamber, wherein the lysing chamber comprises a volume for holding a fluid;
a motor connected to the lysing chamber, wherein the motor is operable to manipulate the lysing chamber;
beads positioned in the volume of the lysing chamber;
a cooling system, wherein the cooling system cools one or more of the fluid and cell protein released from lysed cells in the fluid;
at least one pump for pumping the fluid into the lysing chamber; and
a processor configured to:
send a signal to the at least one pump to automatically pump the fluid into the lysing chamber;
send a signal to the motor to automatically manipulate the lysing chamber;
after a predetermined period of time, send a signal to the motor to automatically stop the manipulation of the lysing chamber; and
send a signal to the at least one pump to automatically pump the cell protein released from lysed cells out of the lysing chamber.

2. The system of claim 1, further comprising a separation system fluidly connected to the lysing chamber, wherein the at least one pump automatically pumps cell protein out of the lysing chamber and to the separation system.

3. The system of claim 1, further comprising a cell growth system fluidly connected to the lysing chamber, wherein the at least one pump pumps the fluid from the cell growth system to the lysing chamber.

4. The system of claim 2, further comprising a protein recovery system fluidly connected to the separation system, wherein the at least one pump pumps cell protein from the separation system to a protein recovery system.

5. The system of claim 1, wherein the system is closed and fluid may move among the cell growth system, the lysing chamber, the separation system, and the protein recovery system without exposure to the atmosphere.

6. A method of lysing cells, the method comprising:
automatically transferring fluid with microorganisms into a lysing chamber;
automatically lysing cells in the fluid by manipulating the lysing chamber which includes beads positioned in the lysing chamber; and
after the manipulating, automatically transferring cell protein from the lysing chamber to a protein recovery system.

7. The method of claim 1, wherein the transferring comprises automatically pumping the fluid into the lysing chamber from a cell growth system.

8. The method of claim 1, further comprising, recovering the cell protein.

9. The method of claim 1, further comprising before the transferring fluid into the lysing chamber, automatically growing the microorganisms in a cell growth system.

10. The method of claim 1, wherein the method is performed in a closed system without exposure to the atmosphere.

11. An automated system for producing protein, the system comprising:
a cell growth system for growing genetically modified microorganisms;
an optical system for determining a concentration of microorganisms in a sample;
a separation system for separating at least a portion of the microorganisms from liquid;
a fluid circulation system that provides fluid communication among the cell growth system, the optical system, and the separation system; and
at least one processor configured to perform one or more steps comprising:
control microorganisms growth conditions in the cell growth system;
control a first flow of fluid from the cell growth system to the optical system;
control a second flow of fluid from the cell growth system to the separation system; and
control a third flow of liquid from the separation system to a container; and
control a fourth flow of separated microorganisms from the separation system.

12. The automated system of claim 11, wherein the separation system comprises a centrifuge.

13. The automated system of claim 12, wherein the separation system further comprises a filter.

14. The automated system of claim 11, wherein the at least one processor is further configured to control the microorganisms growth conditions to optimize the growth of Escherichia coli and/or genetically modified Escherichia coli.

15. The automated system of claim 11, further comprising a cell lysing system for lysing the separated microorganisms to release protein, and wherein the at least one processor is further configured to control the fourth flow of the separated microorganisms from the separation system to the lysing system.

16. The automated system of claim 15, wherein the lysing system comprises:
a lysing chamber, wherein the lysing chamber comprises a volume for holding a fluid;
a motor connected to the lysing chamber, wherein the motor is operable to manipulate the lysing chamber;
beads positioned in the volume of the lysing chamber;
a cooling system, wherein the cooling system cools one or more of the fluid and cell protein released from lysed cells in the fluid; and
at least one pump for pumping fluid into the lysing chamber.

17. The automated system of claim 15, further comprising a protein recovery system for recovering the released protein, and wherein the at least one processor is further configured to control flow of the released protein from the lysing system to the protein recovery system.

18. The automated system of claim 17, wherein the recovery system comprises an affinity column for binding the released protein.

19. The automated system of claim 18, wherein the recovery system further comprises a container for storing an extraction fluid and the released protein after the released protein has been extracted from material in the affinity column using the extraction fluid.

20. The automated system of claim 17, wherein the fluid circulation system is a closed system that provides fluid communication among the cell growth system, the optical system, the separation system, the lysing system, and the protein recovery system.
